(19) United States
(12) Patent Application Publication
(10) Pub. No.: US 2008/0033461 A1
(43) Pub. Date: Feb. 7, 2008

(54) Tow-Dimensional Mesh Implant For Hernia Care

(76) Inventors: Ferdinand Koeckerling, Hannover (DE); Hanngoerg Zimmermann, Goessweinstein (DE); Markus Heinlein, Goessweinstein (DE)

Correspondence Address: MCGLEW & TUTTLE, PC, P.O. BOX 9227, SCARBOROUGH STATION, SCARBOROUGH, NY, 10510-9227, US

(21) Appl. No.: 10/599,837

(22) PCT Filed: Apr. 19, 2005
(86) PCT No.: PCT/EP05/04133

§ 371 Date: Aug. 16, 2007

(30) Foreign Application Priority Data
Apr. 26, 2004 (DE) 102004020469.1

(51) Int. Cl. A61B 17/08

(52) U.S. Cl. 606/151

(57) ABSTRACT

Disclosed is a planar mesh implant for supplying hernia, comprising identical, superimposed mesh layers (1, 2), each of which is provided with a central opening (3, 4) and access slits (5, 6) that are offset relative to one another. The two mesh layers (1, 2) are joined together on one common side of the access slits.

The invention relates to a two-dimensional mesh implant for the care, in particular, of inguinal hernias.

Mesh implants of this type are commonplace in medicine in the most varied configurations and are standard products in the care of hernias. A specific configuration is disclosed for example in WO 00/67663 A1.

Hernia meshes are used in the operative repair of inguinal hernias in particular, in order to achieve an untensioned coverage of the defect to stabilise the abdominal wall. Depending on the type and position of the hernia, it may be necessary to pass a bodily tube, for example the spermatic cord in the case of an inguinal hernia, through the implant. To this end, a passage opening is provided in the mesh layer. Since the bodily tube naturally does not have an end which can be threaded into the passage opening, an insertion slit has to be made in the mesh layer between the outer contour thereof and the central passage opening in order to thereby introduce said tube.

In conventional surgical operations, following the introduction of the tube into the passage opening, this slit is closed by bringing the edges thereof into an overlap position and sewing them together. However, this drawing together means that the mesh implant is deformed, which may hinder a neat position on the abdominal wall. Furthermore, since very thin, light threads are used, mesh implants of this type are not very stable mechanically, which complicates manageability during the operation.

On this basis, the object of the invention is to construct a two-dimensional mesh implant for hernia care such that during implantation, it may be positioned in an easier and neat manner from a surgical operation point of view.

This object is achieved by the features stated in the characterising part of claim 1. The crux of the invention is the formation of the two-dimensional mesh implant from two annular mesh layers which surround a central opening and which each have an access slit, interrupting the annular path, to their central opening. The two mesh layers are superimposed with substantially aligning central openings, the positions of the access slits being offset with respect to one another and, based on the peripheral direction, are rigidly connected only on one common side of the access slits.

The double-layered nature of the mesh implant with a join between the two layers on the one hand provides improved stability of the mesh implant, which is advantageous in particular for straightforward extendability of the implant at its implantation site, for example between the fascia and the abdominal wall. The body tube to be positioned through the mesh implant may be introduced simply by pulling apart the unjoined annular sectors of the two mesh layers and by inserting the tube into the central opening thereof.

Preferred embodiments, other features, details and advantages of the invention are set out in the sub-claims and the following description, in which an embodiment of the subject-matter of the invention will be described in more detail with reference to the accompanying drawings, in which:

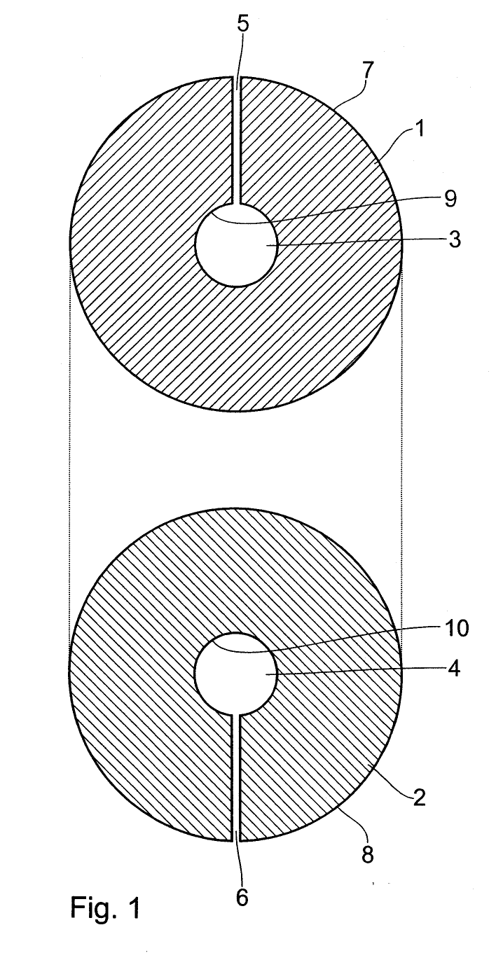

FIG. 1 is a top view, in the manner of an exploded illustration, of the two mesh layers of a mesh implant, and

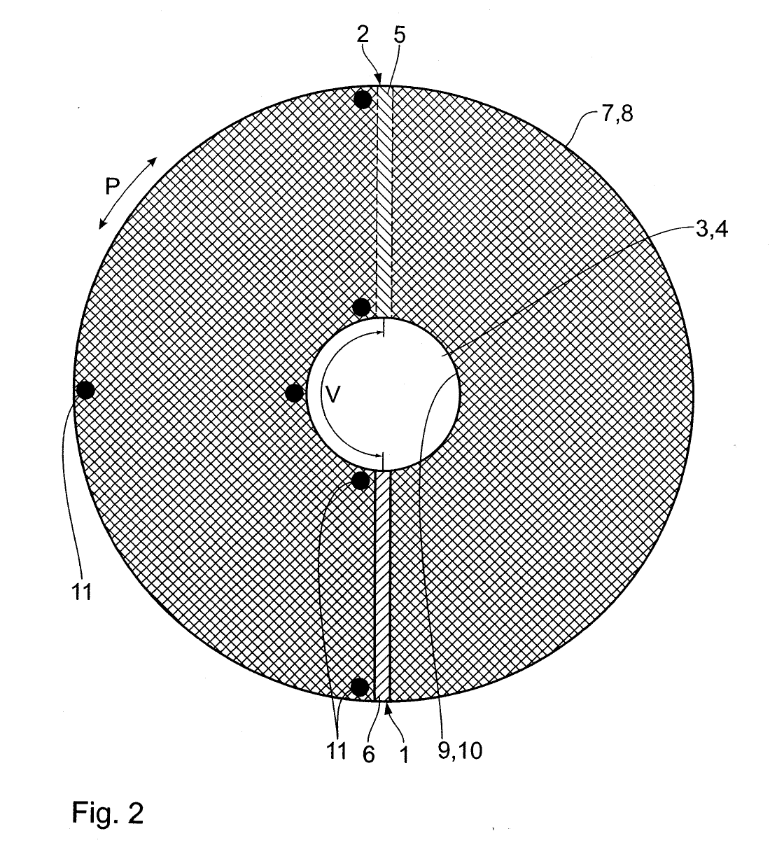

FIG. 2 is a top view of the two-dimensional mesh implant.

As may be seen in particular from FIG. 1, the mesh implant consists of a first mesh layer 1 and a second mesh layer 2 which are identically configured rings having a central opening 3, 4. In the radial direction, the two mesh layers 1, 2 are each provided with an access slit 5, 6 from their peripheral outer edge 7, 8 to the central opening 3, 4.

The two mesh layers 1, 2 consist of a polypropylene monofilament mesh material which is warp knit in atlas lapping with a thread thickness of 100 dtex. The mass per unit area of this layer material for each mesh layer 1, 2 is approximately 60 to 65 g/m² but it may also be selected well below this range. Although it is not shown specifically, the mesh layers 1 have been cut out of a suitable web material by laser cutting.

To produce the complete mesh implant, as shown in a top view in FIG. 2, the two mesh layers 1, 2 thus prefabricated are positioned one on top of the other so that their contours are congruent, although the two access slits 5, 6 are positioned offset with respect to one another by an offset angle V of 180°. On one side, based on the peripheral direction P of the mesh layers 1, 2, that is, on the left-hand side common to the access slits 5, 6, based on FIG. 2, the two mesh layers 1, 2 are rigidly connected at three connection points 11 distributed uniformly over the internal circumferential edges 9, 10 of the central openings 3, 4 or outer edges 7, 8 respectively. The connection points 11 may consist, for example, of seamed points, produced from the same thread material as the mesh layers 1, 2 themselves, or of bonded points, for example of a thermoplastic adhesive.

Although again not shown explicitly in the drawings, after the mesh implant has been made up from the two mesh layers 1, 2, it is provided with a layer of titanium covering the entire surface of the filaments, as a body-compatible coating, by a PACVD process known from the prior art and described in detail, for example, in DE 199 45 299 A. The thickness of the coating is in the region of <2 μm, preferably ranging from approximately 5 to 700 nm.

This continuous metal coating layer on the plastics mesh material significantly improves the fabric compatibility of the mesh implant. This is also promoted by the aforementioned laser cutting process of the mesh layers 1, 2, as this process does not produce any “fraying” with fibre particles becoming loose along the cut edges, but instead results in a cleanly fused peripheral edge region.

1. Two-dimensional mesh implant for hernia care, characterised by a first annular mesh layer (1), surrounding a central opening (3), with an access slit (5), interrupting the annular path, towards the central opening (3), a second annular mesh layer (2) surrounding a central opening (4), also with an access slit (6) interrupting the annular path, towards the central opening (4), the two mesh layers (1, 2) being superimposed with aligning central openings (2, 4) with the positions of the access slits (5, 6) being offset with respect to one another and the two mesh layers being joined together only on one common side of the access slits (5, 6) based on the peripheral direction (P).

2. Mesh implant according to claim 1, characterised in that the two access slits (5, 6) are positioned offset with respect to one another by an angle (V) of 180°.

3. Mesh implant according to either claim 1 or claim 2, characterised in that the two mesh layers (1, 2) have a congruent shape.

4. Mesh implant according to any one of the preceding claims, characterised in that the two mesh layers are joined together by connection points (11) in the form of seamed points or bonded points.

5. Mesh implant according to claim 4, characterised in that the connection points (11) are in each case positioned along the inner circumferential edge (9, 10) of the central opening (3, 4) and along the outer edge (7, 8) of the mesh layers (1, 2).

6. Mesh implant according to any one of the preceding claims, characterised in that it is cut out of a mesh web material, preferably made from polypropylene, using a laser cutting beam.

7. Mesh implant according to any one of the preceding claims, characterised by a metal-containing, continuous, body-compatible coating containing metal.

8. Mesh implant according to claim 7, characterised in that the coating is a titanium-containing coating with a thickness of less than 2 μm, preferably from 5 to 700 nm.
